(19) United States
(12) Patent Application Publication
(10) Pub. No.: US 2010/0068305 A1
(43) Pub. Date: Mar. 18, 2010

(54) MIXTURE FOR USE AS A WOUND DRESSING

(75) Inventors: Rainer Lueck, Tornesch (DE); Swen Neander, Hamburg (DE)

Correspondence Address: Casimir Jones, S.C., 2275 DEMING WAY, SUITE 310, MIDDLETON, WI, 53562, US

(73) Assignee: ERNST MÜHLBAUER GMBH & CO., KG, Norderfriedrichskoog (DE)

(21) Appl. No.: 12/552,865

(22) Filed: Sep. 2, 2009

(30) Foreign Application Priority Data
Sep. 3, 2008 (DE) 102008045523.7

(51) Int. Cl. A61K 33/08; A61P 19/04

(52) U.S. Cl. 424/693

(57) ABSTRACT

A subject matter of the invention is a mixture, which comprises:
a) wax esters;
b) at least one metal hydroxide;
c) optionally radio-opaque filler;
   the proportion of the components a), b) and c) in the mixture being at least 93% by weight.

The invention relates to a mixture which is suitable as wound dressing and to the use thereof as wound dressing. Periodontitis is an inflammatory disease of the periodontic apparatus. A therapeutic starting point is curettage, in which inflamed tissue and/or contaminants are mechanically removed. After curettage of a periodontal pocket, injuries to the tissue of the tooth (dentin) and of the gingiva are accordingly often present.

Oil mixtures comprising calcium hydroxide for the care of periodontal pockets in inflammatory disease, which oil mixtures are to be of vegetable or synthetic origin, are known from EP 1 224 924 A2. Glycerides of oleic acid are preferred main component. An oil mixture which essentially corresponds to that of neatsfoot oil is claimed.

Oils are esters formed from fatty acids and glycerol. Oils generally have a tendency to saponify with metal hydroxides. Mixtures, such as those of EP 1 224 924 A2, are not stable on storage. The calcium hydroxide component and the oil component of such preparations are accordingly stored separately and only mixed immediately before use. This can result in applicational failings and does not guarantee the optimum (homogeneity, mixing ratio) mixture.

Calcium hydroxide preparations free from neatsfoot oil which are suitable as wound dressing are known from EP 0 908 169 A2, which preparations comprise 10-50% by weight of paraffin hydrocarbons and other hydrocarbon-based synthetic waxes. Vegetable oils, fats or waxes can be added to adjust the consistency. The oils, fats or waxes having a tendency to saponify are stabilized in the overall mixture by the paraffin hydrocarbon or other hydrocarbon-based synthetic waxes present.

It is an object of the invention to create a mixture which is stable in storage in the ready-for-use condition and which advantageously can also be used in the treatment of wounds after a periodontitis treatment.

A mixture according to the invention comprises:
a) wax esters;
b) at least one metal hydroxide;
c) optionally radio-opaque filler;
   the proportion of the components a), b) and c) in the mixture being at least 93% by weight.

A few terms used in the context of the invention should first be explained. Wax esters are esters of long-chain carboxylic acids with long-chain alcohols. Generally, naturally occurring waxes are wax esters in this sense. Suitable wax esters are, for example, disclosed in Ullmann's Encyclopedia of Industrial Chemistry, 6th edition, Volume A 39, page 135 ff. Reference may be made there in particular to page 142 ff. (vegetable waxes) and page 150 ff. (animal waxes).

The term “wound dressing” is to be understood in the broad sense in the context of the invention and comprises in particular uses in oral surgery, implantology, traumatology or the like, in which the mixture according to the invention is applied to or in bone traumas, such as, for example, fracture surfaces, drill holes, cavities or the like. The term includes, for example, the root filling and also the use in the context of the fixing of temporary tooth stump coverings.

The term includes in particular compositions for the care of wounds after surgery or invasive treatment of the periodontic apparatus, for example curettage for the treatment of periodontitis.

Surprisingly, it has been established that mixtures according to the invention are stable on storage in the ready-for-use condition. This is in particular therefore surprising, since wax esters have a tendency to saponify.

Accordingly, in the state of the art, the stability of the overall mixture was achieved through a substantial proportion of nonsaponifying hydrocarbon-based synthetic waxes.

The possible dispensing according to the invention with such synthetic waxes makes it possible for the mixtures according to the invention to be largely or completely biodegradable. This advantageously allows in particular care of periodontal pockets after a periodontitis treatment since the mixtures according to the invention are effective there over a relatively long period of time and are decomposed or, in the remedying of tissue injuries, for example in the dentin, are incorporated. Bone formation involves first the synthesis of organic tissue (collagen synthesis) and subsequently thereto the incorporation, mediated by the “matrix vesicles”, of mineral substance in the organic matrix. The connective tissue protein collagen is the main component of the organic substance of the bone. The protein consists of three helically coiled polypeptide chains, the amino acid sequences of which can vary, which results in a great variety of individual collagen types. An extraordinarily high mechanical strength of the collagen fiber is common to all collagen types. This strength is produced by a multitude of intra- and intermolecular linkages of the peptide chains, which in this way form the dense collagen fiber network of the connective tissue.

These linkages arise through the oxidation of the lysine and the subsequent reaction of the aldehyde formed with free amino groups of neighboring chains. In addition, hydrogen bonds and ester linkages with sugar residues occur. The bony tissue is formed by the incorporation of mineral substances (hydroxyapatite, calcium phosphate) in this network. The mixtures according to the invention exhibit good regeneration-promoting properties which contribute to tissue regeneration.

In the care of wounds from periodontal pockets, it is advantageous for the mixture to exhibit a sufficiently pasty consistency, so that it can remain in the periodontal pocket over a certain period of time. The extensive or complete biodegradability causes the establishment of bony tissue in the periodontal pocket to be stimulated and not to be hindered by nondecomposable synthetic substances.

Preference is given to the wax esters chosen from the group consisting of wax esters of the formula

R1—O—CO—R2

in which R1 and R2, which are identical or different, are straight-chain or branched alkyl or alkenyl groups with from 4 to 42 carbon atoms. In this formula, R1 is the alcohol residue and R2 is the carboxylic acid residue of the fatty acid. R1 and R2 are preferably residues of naturally occurring fatty acids or fatty alcohols.

The fatty alcohol residue R1 preferably exhibits from 16 to 24 carbon atoms and the fatty acid residue R2 preferably exhibits from 18 to 24 carbon atoms. In total, the wax esters preferably exhibit altogether from 36 to 46 carbon atoms. Use is preferably made of wax esters which are liquid at ambient temperature, since in this way pasty mixtures with high metal hydroxide contents can be prepared. Preferably, the wax esters comprise jojoba oil or are essentially composed of jojoba oil.

Jojoba oil is composed essentially of glyceride-free fatty acid esters with from 36 to 46 carbon atoms (C₃₆₋₄₆). These are essentially formed from monounsaturated eicosenoic acid (C₂₀, approximately 70%), docosenoic acid (C₂₂, approximately 20%) and additional fatty acids with long-chain wax monoalcohols (C₁₆₋₂₄).

Jojoba oil is preferred, since it is a natural wax which is liquid at ambient temperature. Accordingly, the preferred pasty consistency can be prepared, even with high metal hydroxide contents, without additional additives, such as oils or synthetic waxes, being necessary.

It is possible in the context of the invention, but less preferred, for the mixture to comprise a small proportion (maximum 7% by weight) of additional additives, such as paraffin hydrocarbons or other hydrocarbon-based synthetic waxes.

Depending on the field of application, the composition can comprise additional waxes, petroleum jellies, paraffin hydrocarbons which are liquid at ambient temperature, such as paraffinum liquidum or paraffinum perliquidum, and the like, and plant and/or animal oils and/or fats, and also wax alcohols, the proportion of which is preferably less than 10% by weight, more preferably less than 5% by weight, of the powder-free part of the mixture.

Furthermore, it is optionally possible, in particular when used as root filling substance, for well-known radio-opaque filling powders customary in dentistry to be present. The choice thereof depends mainly on their toxicological harmlessness, a high availability and low cost. Inert, physiologically harmless, radio-opaque filling powders, such as powders of glasses or salts of heavy metals or rare earth metals, are preferably present, for example the dental glass powders from Schott AG, Mainz. Particularly suitable heavy metals or rare earth metals are, for example, yttrium, ytterbium or zirconium, cerium, tin, zinc, barium, strontium, bismuth, tungsten and molybdenum. Particularly suitable salts are oxides, sulfates, phosphates or halides. Particularly preferably, barium sulfate is present.

The radio-opaque fillers preferably produce a radio-opacity of the mixture according to ISO 4049 of at least 50% aluminum, preferably at least 100% aluminum, more preferably at least 200% aluminum.

Furthermore, additives, such as fillers, fluoride, apatite, oxidation inhibitors, inter alia, can be added.

Preferably, the proportion of wax esters (component a), metal hydroxides (component b) and radio-opaque fillers (component c) in the overall mixture is at least 95% by weight; more preferred lower limits are 98 and 99% by weight. More preferably, the mixture is essentially free from nonbiodegradable ingredients.

The proportion of the component a) in the mixture is preferably from 10 to 60% by weight, more preferably from 15 to 50% by weight, more preferably from 20 to 50% by weight, more preferably from 25 to 50% by weight, more preferably from 30 to 45% by weight. The proportion of the component b) in the mixture is preferably from 40 to 90% by weight, more preferably from 50 to 85% by weight, more preferably from 50 to 80% by weight, more preferably from 50 to 75% by weight, more preferably from 55 to 70% by weight; if component c) is present, more preferably from 20 to 45% by weight, more preferably from 25 to 40% by weight, more preferably from 25 to 35% by weight. The proportion of the optional component c) in the mixture is from 0 to 50% by weight, preferably from 20 to 35% by weight.

Alkali metal and alkaline earth metal hydroxides are preferred as metal hydroxides; calcium hydroxide is particularly preferred. A mixture of calcium hydroxide with wax esters brings about a delayed release of the alkalinity which results in a bactericidal action and stimulates collagen regeneration in vivo.

Particular preference is given to wound dressings consisting exclusively of calcium hydroxide and an animal and/or vegetable wax (wax ester). Very particularly preferably, the wound dressing consists of calcium hydroxide and jojoba oil. Limiting to the active component calcium hydroxide and the generally well-tolerated jojoba oil, which is per se antiinflammatory, as paste formers reduces the danger of the occurrence of side effects, such as allergic reactions, contact dermatitis or the like (even of a local nature).

Additional suitable natural vegetable and animal waxes are, for example, carnauba wax, candelilla wax, ouricury wax, beeswax, inter alia.

Synthetic wax esters or mixtures of wax esters of the formula R1—O—CO—R2, R1 and R2 being able to be identical or different and representing alkyl or alkenyl groups with from 4 to 42 carbon atoms, are also suitable.

The mixtures can then in particular be used if a very high content of calcium hydroxide is needed, radio-opacity is not required, the absence of all extraneous components (fillers, synthetic additives) is desirable and also nonbiodegradable components, such as petroleum jellies (paraffin hydrocarbons), should not be present in the composition. This is the case in particular in the region of the periodontic apparatus.

Mixtures according to the invention are preferably stable in storage for at least one year, more preferably at least two years, at ambient temperature (22° C.)

Another subject matter of the invention is a mixture described above for use as medicament (first medicinal indication) and for use as wound dressing (second medicinal indication). Another subject matter of the invention is the use of such a mixture as wound dressing or for preparing a wound dressing.

The invention is explained below with the use of examples.

### EXAMPLE 1

### (According to the Invention)

295.4 g of calcium hydroxide were homogeneously mixed with 180.6 g of jojoba oil. Subsequently, the paste was degassed. The paste thus obtained had a creamy consistency and is accordingly suitable, for example, for the filling of periodontal pockets after curettage.

The pH value was measured to assess the effectiveness with regard to the regeneration of the (bony) tissue (stimulation of collagen regeneration). This should not exceed a value of approximately 11, preferably 10.5, in order to stimulate collagen regeneration.

The measurement was performed in an aqueous solution with a pH electrode in the system buffered at pH 7. To prepare the buffer solution, 68 g of imidazole were diluted with distilled water and the solution adjusted to pH 7 using 0.1N HCl. The solution was subsequently made up to 1 l with distilled water.

30 ml of the buffer solution were poured over 6 g of the calcium hydroxide paste prepared and the change in the pH value over a period of time of four hours was measured. In this connection, values of between 9.5 and 10.5 resulted. The paste is accordingly very well-suited for use in periodontology.

To determine the stability on storage, dental syringes

(Luer Lock needle (1 mm), Superlux®—calcium hydroxide liner, DMG Chemisch-Pharmazeutische Fabrik GmbH) were filled with 2 ml of the paste and stored at ambient temperature and in a drying cupboard at 33° C. or 40° C. Subsequently, the applicability of the mixture from the syringe via a needle, and also the consistency of the expressed paste, was investigated in each case. The results are reproduced in table 1.

TABLE 1

| Storage conditions | Consistency | Applicability |
|---|---|---|
| Without storage | Creamy | Easy |
| 3 months at 23° C. | Creamy | Easy |
| 3 months at 33° C. | Creamy | Easy |
| 3 months at 40° C. | Creamy | Easy |
| 30 months at 23° C. | Creamy | Easy |

### EXAMPLE 2

### (According to the Invention)

300 g of the powder component of a commercially available calcium hydroxide preparation (Gangraena-Merz® N (1:1 mixture of calcium hydroxide and barium sulfate), Merz Dental GmbH) were homogeneously mixed with 151 g of jojoba oil. Subsequently, the paste was degassed. To determine the stability on storage, dental syringes (Luer Lock needle (1 mm), Superlux®—calcium hydroxide liner, DMG Chemisch-Pharmazeutische Fabrik GmbH) were filled with 2 ml of the paste and stored at ambient temperature and in a drying cupboard at 33° C. or 40° C. Subsequently, the applicability of the mixture from the syringe via a needle, and also the consistency of the expressed paste, was investigated in each case. The results are reproduced in table 2.

TABLE 2

| Storage conditions | Consistency | Applicability |
|---|---|---|
| Without storage | Creamy | Easy |
| 3 months at 23° C. | Creamy | Easy |
| 3 months at 33° C. | Creamy | Easy |
| 3 months at 40° C. | Creamy | Easy |
| 30 months at 23° C. | Creamy | Easy |

### EXAMPLE 3

### (Comparative Example)

40 g of the powder component of a commercially available calcium hydroxide preparation (Gangraena-Merz® N powder (1:1 mixture of calcium hydroxide and barium sulfate), Merz Dental GmbH) were homogeneously mixed with 21 g of the oil component of a commercially available calcium hydroxide preparation (Gangraena-Merz® N oil (mixture of fatty acid triglycerides), Merz Dental GmbH). Subsequently, the paste was degassed. To determine the stability on storage, dental syringes (Luer Lock needle (1 mm), Superlux®—calcium hydroxide liner, DMG Chemisch-Pharmazeutische Fabrik GmbH) were filled with 2 ml of the paste and stored at ambient temperature and in a drying cupboard at 33° C. or 40° C. Subsequently, the applicability of the mixture from the syringe via a needle, and also the consistency of the expressed paste, was investigated in each case. The results are reproduced in table 3.

TABLE 3

| Storage conditions | Consistency | Applicability |
|---|---|---|
| Without storage | Creamy | Easy |
| 3 months at 23° C. | Clearly more solid | Difficult |
| 3 months at 33° C. | Hardened | Cannot be applied |
| 3 months at 40° C. | Hardened | Cannot be applied |
| 30 months at 23° C. | Hardened | Cannot be applied |

### EXAMPLE 4

### (Comparative Example)

40 g of calcium hydroxide were homogeneously mixed with 21 g of the oil component of a commercially available calcium hydroxide preparation (Gangraena-Merz® N oil (mixture of fatty acid triglycerides), Merz Dental GmbH). Subsequently, the paste was degassed. To determine the stability on storage, dental syringes (Luer Lock needle (1 mm), Superlux®—calcium hydroxide liner, DMG Chemisch-Pharmazeutische Fabrik GmbH) were filled with 2 ml of the paste and stored at ambient temperature and in a drying cupboard at 33° C. or 40° C. Subsequently, the applicability of the mixture from the syringe via a needle, and also the consistency of the expressed paste, was investigated in each case. The results are reproduced in table 4.

TABLE 4

| Storage conditions | Consistency | Applicability |
|---|---|---|
| Without storage | Creamy | Easy |
| 3 months at 23° C. | Clearly more solid | Difficult |
| 3 months at 33° C. | Hardened | Cannot be applied |
| 3 months at 40° C. | Hardened | Cannot be applied |
| 30 months at 23° C. | Hardened | Cannot be applied |

1. Mixture, which comprises:
a) wax esters;
b) at least one metal hydroxide;
c) optionally radio-opaque filler;
the proportion of the components a), b) and c) in the mixture being at least 93% by weight.

2. Mixture according to claim 1, characterized in that the wax esters are chosen from the group consisting of wax esters of the formula
R1—O—CO—R2
in which R1 and R2, which are identical or different, are straight-chain or branched alkyl or alkenyl groups with from 4 to 42 carbon atoms.

3. Mixture according to claim 2, characterized in that R1 exhibits from 16 to 24 carbon atoms and R2 exhibits from 18 to 24 carbon atoms.

4. Mixture according to claim 2 or 3, characterized in that the wax esters exhibit altogether from 36 to 46 carbon atoms.

5. Mixture according to one of claims 1 to 4, characterized in that the wax esters are liquid at ambient temperature.

6. Mixture according to one of claims 1 to 5, characterized in that the wax esters comprise jojoba oil or, preferably, are essentially composed of jojoba oil.

7. Mixture according to one of claims 1 to 6, characterized in that the proportion of the components a) and b) and radio-opaque fillers (component c)) in
the overall mixture is at least 95% by weight, preferably at least 98% by weight, more preferably at least 99% by weight.

8. Mixture according to one of claims 1 to 7, characterized in that the proportion of the component a) in the mixture is from 10 to 70% by weight, preferably from 10 to 60% by weight, more preferably from 15 to 50% by weight, more preferably from 25 to 50% by weight, more preferably from 30 to 45% by weight, more preferably from 40 to 50% by weight.

9. Mixture according to one of claims 1 to 8,
characterized in that the proportion of the component b) in the mixture is from 30 to 90% by weight, preferably from 40 to 90% by weight, more preferably from 50 to 85% by weight, more preferably from 50 to 80% by weight, more preferably from 50 to 75% by weight, more preferably from 55 to 70% by weight.

10. Mixture according to one of claims 1 to 8, characterized in that it comprises up to 50% by weight, preferably from 20 to 35% by weight, of the component c) and in that the proportion of the component b) is from 20 to 45% by weight, preferably from 25 to 40% by weight, more preferably from 25 to 35% by weight.

11. Mixture according to one of claims 1 to 9, characterized in that the component b) is chosen from the group consisting of alkali metal and alkaline earth metal hydroxides.

12. Mixture according to claim 11, characterized in that the component b) is calcium hydroxide.

13. Mixture according to one of claims 1 to 12, characterized in that it is essentially free from nonbiodegradable ingredients.

14. Mixture according to one of claims 1 to 13 for use as a medicament.

15. Mixture according to one of claims 1 to 13 for use as wound dressing.

16. Use of a mixture according to one of claims 1 to 13 for preparing a wound dressing.
